(19) United States
(12) Patent Application Publication
(10) Pub. No.: US 2026/0083715 A1
(43) Pub. Date: Mar. 26, 2026

(54) COMPOSITION FOR ENHANCING IMMUNITY CONTAINING AMIDE-BASED COMPOUND

(71) Applicant: AMOREPACIFIC CORPORATION, Seoul (KR)

(72) Inventors: Eun Jeong CHOI, Yongin-si (KR); Geunhyuk JANG, Yongin-si (KR); Hyunjung CHOI, Yongin-si (KR); Hyoung June KIM, Yongin-si (KR); Wonseok PARK, Yongin-si (KR); Heungsoo BAEK, Yongin-si (KR); Jaewon YOU, Yongin-si (KR)

(73) Assignee: AMOREPACIFIC CORPORATION, Seoul (KR)

(21) Appl. No.: 19/335,570

(22) Filed: Sep. 22, 2025

(30) Foreign Application Priority Data
Sep. 25, 2024 (KR) 10-2024-0130069

(51) Int. Cl. A61K 31/445; A61K 31/197; A61K 31/40; A61P 37/04

(52) U.S. Cl. CPC A61K 31/445; A61K 31/197; A61K 31/40; A61P 37/04

(57) ABSTRACT

The present specification relates to a composition containing a novel amide-based compound derived from an amino acid structure such as valine, leucine, phenylalanine, proline, or pipecolic acid, and the composition can exhibit an immune-enhancing effect by activating the phagocytosis of macrophages, which aids in removing pathogenic bacteria as an important part of the human immune system.

### CROSS REFERENCE TO RELATED APPLICATION

The present application claims priority to Korean Patent Application No. 10-2024-0130069, filed Sep. 25, 2024, the entire contents of which are hereby incorporated by this reference.

### BACKGROUND OF THE INVENTION

### Field of the Invention

The present specification discloses a composition for enhancing immunity containing an amide-based compound.

### Description of the Related Art

Macrophages are an important part of the human immune system, and play a key role in removing pathogenic bacteria from within the skin. Since infection by pathogenic bacteria may cause various skin problems such as acne, inflammation, and skin diseases, the phagocytosis of macrophages, which aids in removing pathogenic bacteria, is one of the important defense mechanisms that protect the skin from pathogenic bacteria.

However, to date, there has been little progress in identifying substances that enhance the phagocytosis of macrophages.

### SUMMARY OF THE INVENTION

One aspect of the present disclosure aims to provide a composition for enhancing immunity, containing a novel compound derived from an amino acid structure, such as valine, leucine, phenylalanine, proline, or pipecolic acid.

In one aspect, the present specification provides a composition for enhancing immunity, containing, as an active ingredient, a compound represented by the following Chemical Formula 1, a stereoisomer thereof, a pharmaceutically acceptable salt thereof, a hydrate thereof, or a solvate thereof.

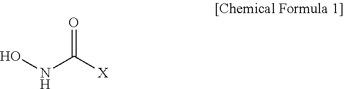

In Chemical Formula 1,
X is represented by any one of the following Chemical Formulae 1-1 to 1-5,

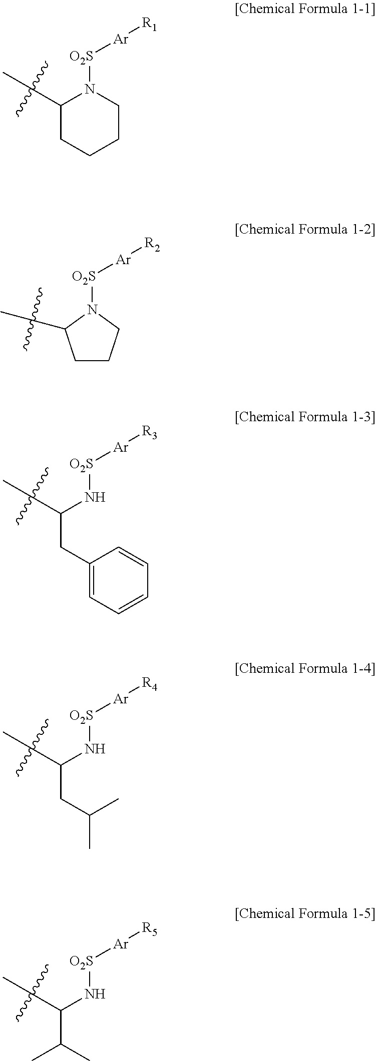

in Chemical Formula 1-1,
Ar is phenyl unsubstituted or substituted with one to three R₁,
R₁ is hydrogen, a phenyl group, a C1-C4 alkoxy group, a C1-C4 alkyl group, a fluoro group, an adamantane group, an acetylamino group, or a hydroxyiminoethyl group,
when there are more than one R₁ residue, all R₁ residues are the same as each other,
in Chemical Formula 1-2,
Ar is phenyl substituted with one R₂,
R₂ is a C1-C4 alkyl group, a C1-C4 alkoxy group, an adamantane group, an acetyl group, or an acetylamino group,
in Chemical Formula 1-3,
Ar is phenyl substituted with one to three R₃ or unsubstituted naphthalene,
R₃ is hydrogen, a C1-C4 alkyl group, or a phenyl group, when there are more than one R₃ residue, all R₃ residues are the same as each other,
in Chemical Formula 1-4,
Ar is phenyl unsubstituted or substituted with one to three R₄,
R₄ is hydrogen, a phenyl group, a C1-C4 alkyl group, or a C1-C4 alkoxy group,
when there are more than one R₄ residue, all R₄ residues are the same as each other,
in Chemical Formula 1-5,
Ar is phenyl substituted with one to three R₅,
R₅ is a C1-C4 alkyl group or a phenyl group, and
when there are more than one R₅ residue, all R₅ residues are the same as each other.

In one aspect, a composition containing the novel amide-based compound disclosed in the present specification can activate the phagocytosis of macrophages to exhibit an immune enhancing effect.

### BRIEF DESCRIPTION OF THE DRAWINGS

FIGS. 1 and 2 show the results of observing the phagocytosis-activating effect of a composition according to an example of the present disclosure.

### DETAILED DESCRIPTION OF THE INVENTION

Hereinafter, the present disclosure will be described in more detail through the following Examples. However, the following Examples are provided only for illustrative purposes to facilitate the understanding of the present disclosure, and the purview and scope of the present disclosure is not limited thereto.

In exemplary embodiments of the present disclosure, provided is a composition for enhancing immunity, containing, as an active ingredient, a compound represented by the following Chemical Formula 1, a stereoisomer thereof, a pharmaceutically acceptable salt thereof, a hydrate thereof, or a solvate thereof:

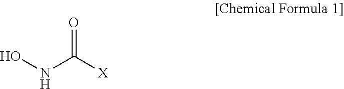

In Chemical Formula 1,
X is represented by any one of the following Chemical Formulae 1-1 to 1-5,

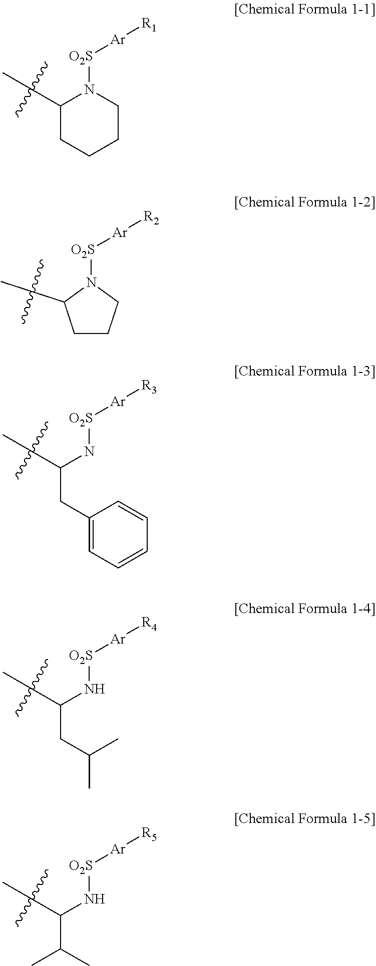

in Chemical Formula 1-1,
Ar is phenyl unsubstituted or substituted with one to three R₁,
R₁ is hydrogen, a phenyl group, a C1-C4 alkoxy group, a C1-C4 alkyl group, a fluoro group, an adamantane group, an acetylamino group, or a hydroxyiminoethyl group,
when there are more than one R₁ residue, all R₁ residues are the same as each other,
in Chemical Formula 1-2,
Ar is phenyl substituted with one R₂,
R₂ is a C1-C4 alkyl group, a C1-C4 alkoxy group, an adamantane group, an acetyl group, or an acetylamino group,
in Chemical Formula 1-3,
Ar is phenyl substituted with one to three R₃ or unsubstituted naphthalene,
R₃ is hydrogen, a C1-C4 alkyl group, or a phenyl group,
when there are more than one R₃ residue, all R₃ residues are the same as each other,
in Chemical Formula 1-4,
Ar is phenyl unsubstituted or substituted with one to three R₄,
R₄ is hydrogen, a phenyl group, a C1-C4 alkyl group, or a C1-C4 alkoxy group,
when there are more than one R₄ residue, all R₄ residues are the same as each other, in Chemical Formula 1-5,
Ar is phenyl substituted with one to three R₅,
R₅ is a C1-C4 alkyl group or a phenyl group, and
when there are more than one R₅ residue, all R₅ residues are the same as each other.

For example, in Chemical Formulae 1-1 to 1-5, R₁ to R₅ may each be substituted at the para position of Ar.

In the present specification, the alkyl group may be a straight or branched chain, for example, methyl, ethyl, propyl, n-propyl, isopropyl, butyl, n-butyl, isobutyl, tert-butyl, or sec-butyl.

In the present specification, the alkoxy group may be a straight, branched or cyclic chain, for example, methoxy, ethoxy, n-propoxy, isopropoxy, i-propyloxy, n-butoxy, isobutoxy, tert-butoxy, or sec-butoxy.

In the present specification, the term “isomer” includes not only optical isomers (for example, essentially pure enantiomers, essentially pure diastereoisomers, or mixtures thereof), but also conformation isomers (that is, isomers that differ only in the angle of one or more chemical bonds), position isomers (particularly tautomers), or geometric isomers (for example, cis-trans isomers).

In the present specification, the term “essentially pure,” when used in connection with, for example, enantiomers or diastereoismers, means that a specific compound, for example, an enantiomer or diastereoisomer, is present in an amount of about 90% or more, preferably about 95% or more, more preferably about 97% or more or about 98% or more, still more preferably about 99% or more, and even more preferably about 99.5% or more (w/w).

In the present specification, the term “pharmaceutically acceptable” means that it can be or has been approved by the government or equivalent regulatory body as being suitable for use in animals, and more particularly, in humans, by avoiding significant toxic effects when used in conventional medicinal dosages, or is listed in a pharmacopoeia or recognized in other general pharmacopoeias.

In the present specification, the term “pharmaceutically acceptable salt” means a salt according to an aspect of the present disclosure which is pharmaceutically acceptable and has the desired pharmacological activity of a parent compound. The salt may include (1) an acid addition salt formed by an inorganic acid such as hydrochloric acid, hydrobromic acid, sulfuric acid, nitric acid, phosphoric acid, and the like; or formed by an organic acid such as acetic acid, propionic acid, hexanoic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethane sulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid, and muconic acid; or (2) a salt formed when acidic protons present in a parent compound are substituted.

In the present specification, the term “hydrate” refers to a compound to which water is bonded, and is a broad concept that includes inclusion compounds which do not have a chemical bonding force between water and the compound.

In the present specification, the term “solvate” refers to a higher order compound formed between molecules or ions of a solute and molecules or ions of a solvent.

In other exemplary embodiments of the present disclosure, provided is a method for enhancing immunity, the method including administering an effective amount of a composition containing, as an active ingredient, the above-described compound represented by Chemical Formula 1, a stereoisomer thereof, a pharmaceutically acceptable salt thereof, a hydrate thereof, or a solvate thereof to a subject in need thereof.

In other exemplary embodiments of the present disclosure, provided is a use of the above-described compound represented by Chemical Formula 1, a stereoisomer thereof, a pharmaceutically acceptable salt thereof, a hydrate thereof, or a solvate thereof, for preparing a composition for enhancing immunity.

In other exemplary embodiments of the present disclosure, provided is a non-therapeutic use of the above-described compound represented by Chemical Formula 1, a stereoisomer thereof, a pharmaceutically acceptable salt thereof, a hydrate thereof, or a solvate thereof, for enhancing immunity.

In an embodiment, the compound represented by Chemical Formula 1 may be any one selected from the following compounds:
N-hydroxy-1-(phenylsulfonyl) piperidine-2-carboxamide,
1-([1, l′-biphenyl]-4-ylsulfonyl)-N-hydroxypiperidine-2-carboxamide,
1-((4-(tert-butyl)phenyl) sulfonyl)-N-hydroxypiperidine-2-carboxamide,
1-((4-butoxyphenyl) sulfonyl)-N-hydroxypiperidine-2-carboxamide,
N-hydroxy-1-(mesitylsulfonyl) piperidine-2-carboxamide,
2-([1, l′-biphenyl]-4-sulfonamido)-N-hydroxy-3-phenylpropanamide,
2-((4-(tert-butyl)phenyl) sulfonamido)-N-hydroxy-3-phenylpropanamide,
N-hydroxy-3-phenyl-2-((2,4,6-trimethylphenyl) sulfonamido) propanamide,
N-hydroxy-2-(naphthalene-2-sulfonamido)-3-phenylpropanamide,
2-([1,1′-biphenyl]-4-sulfonamido)-N-hydroxy-4-methylpentanamide,
2-((4-(tert-butyl)phenyl) sulfonamido)-N-hydroxy-4-methylpentanamide,
2-((4-butoxyphenyl) sulfonamido)-N-hydroxy-4-methylpentanamide,
N-hydroxy-4-methyl-2-((2,4,6-trimethylphenyl) sulfonamido) pentanamide,
2-([1,1′-biphenyl]-4-sulfonamido)-N-hydroxy-3-methylbutanamide,
2-((4-(tert-butyl)phenyl) sulfonamido)-N-hydroxy-3-methylbutanamide,
N-hydroxy-3-methyl-2-((2,4,6-trimethylphenyl) sulfonamido) butanamide,
1-((4-fluorophenyl) sulfonyl)-N-hydroxypiperidine-2-carboxamide,
1-((4-((3r,5r,7r)-adamantan-1-yl)phenyl) sulfonyl)-N-hydroxypiperidine-2-carboxamide,
N-hydroxy-1-((4-propylphenyl) sulfonyl) piperidine-2-carboxamide,
1-((4-acetamidophenyl) sulfonyl)-N-hydroxypiperidine-2-carboxamide,
1-((4-(tert-butyl)phenyl) sulfonyl)-N-hydroxypyrrolidine-2-carboxamide,
1-((4-butoxyphenyl) sulfonyl)-N-hydroxypyrrolidine-2-carboxamide,
1-((4-((3r,5r, 7r)-adamantan-1-yl)phenyl) sulfonyl)-N-hydroxypyrrolidine-2-carboxamide,
N-hydroxy-1-((4-propylphenyl) sulfonyl) pyrrolidine-2-carboxamide,
1-((4-acetylphenyl) sulfonyl)-N-hydroxypyrrolidine-2-carboxamide,
N-hydroxy-1-((4-(1-(hydroxyimino)ethyl)phenyl) sulfonyl) piperidine-2-carboxamide,
N-hydroxy-4-methyl-2-(phenylsulfonamido) pentanamide, and
1-((4-acetamidophenyl) sulfonyl)-N-hydroxypyrrolidine-2-carboxamide.

In an embodiment, the compound represented by Chemical Formula 1 may be represented by any one of the following chemical formulae:

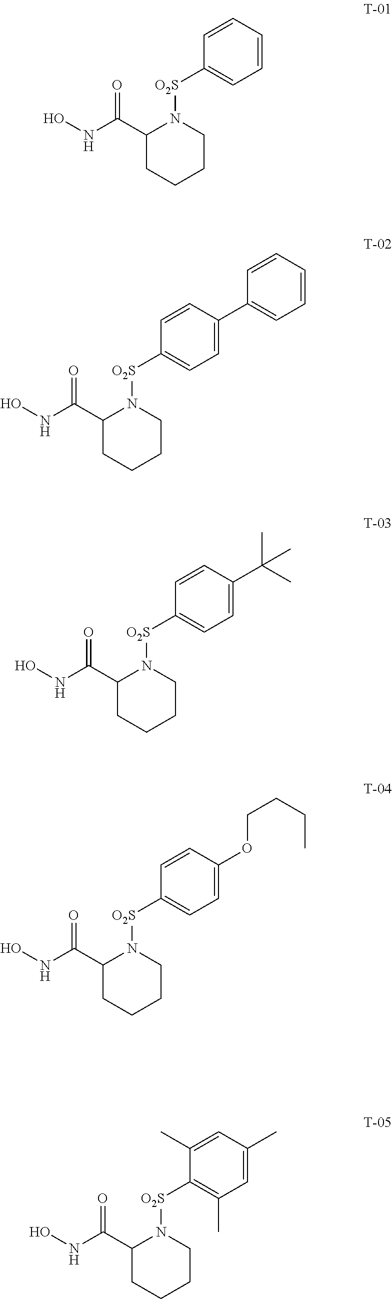 [SMILES: O=C(NO)C1CCCCN1S(=O)(=O)c1ccccc1 ; O=C(NO)C1CCCCN1S(=O)(=O)c1ccc(-c2ccccc2)cc1 ; CC(C)(C)c1ccc(S(=O)(=O)N2CCCCC2C(=O)NO)cc1 ; CCCCOc1ccc(S(=O)(=O)N2CCCCC2C(=O)NO)cc1 ; Cc1cc(C)c(S(=O)(=O)N2CCCCC2C(=O)NO)c(C)c1]

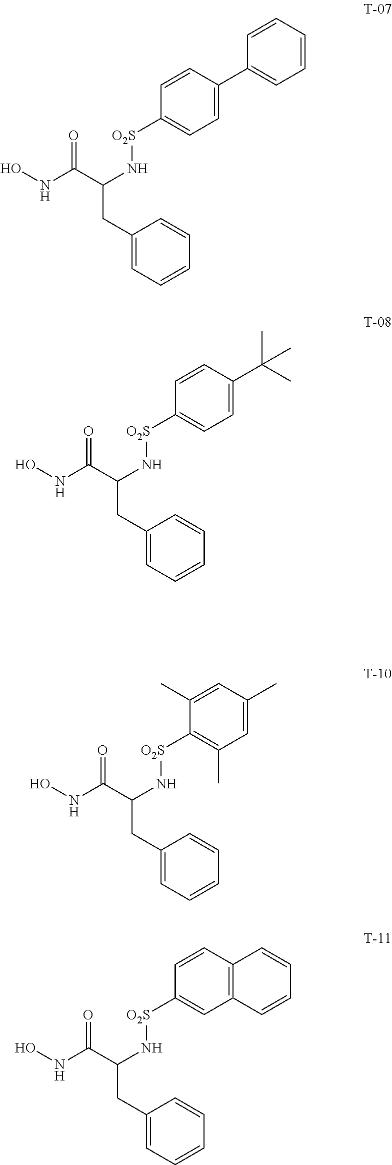 [SMILES: O=C(NO)C(Cc1ccccc1)NS(=O)(=O)c1ccc(-c2ccccc2)cc1 ; CC(C)(C)c1ccc(S(=O)(=O)NC(Cc2ccccc2)C(=O)NO)cc1 ; Cc1cc(C)c(S(=O)(=O)NC(Cc2ccccc2)C(=O)NO)c(C)c1 ; O=C(NO)C(Cc1ccccc1)NS(=O)(=O)c1ccc2ccccc2c1]

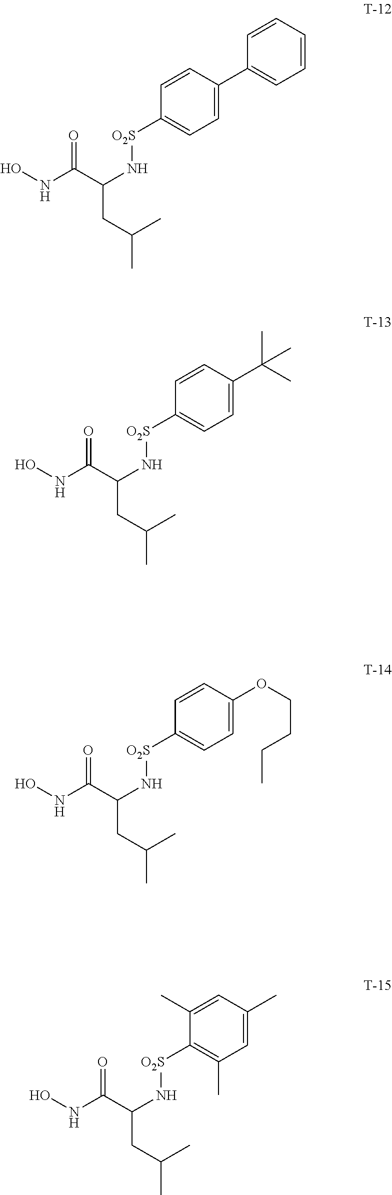 [SMILES: CC(C)CC(NS(=O)(=O)c1ccc(-c2ccccc2)cc1)C(=O)NO ; CC(C)CC(NS(=O)(=O)c1ccc(C(C)(C)C)cc1)C(=O)NO ; CCCCOc1ccc(S(=O)(=O)NC(CC(C)C)C(=O)NO)cc1 ; Cc1cc(C)c(S(=O)(=O)NC(CC(C)C)C(=O)NO)c(C)c1]

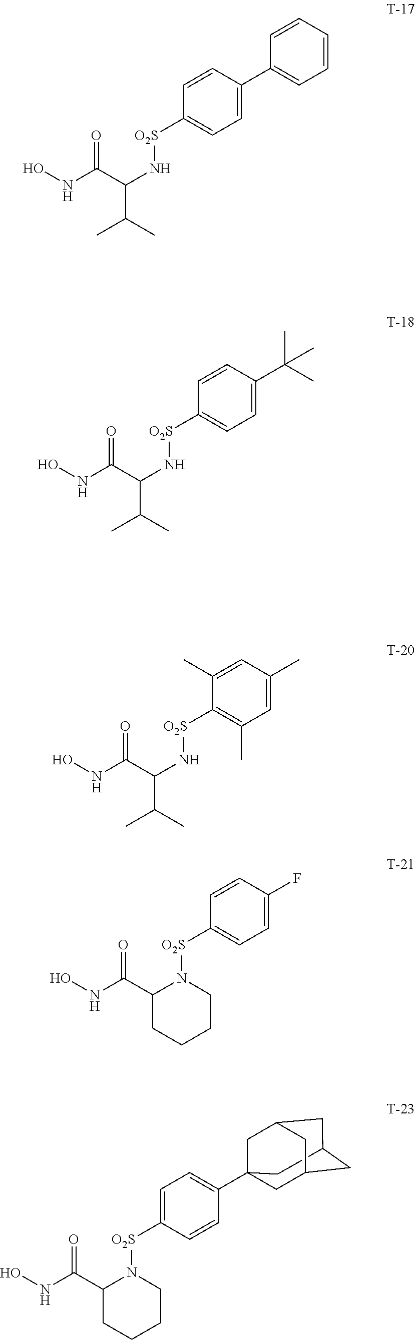 [SMILES: CC(C)C(NS(=O)(=O)c1ccc(-c2ccccc2)cc1)C(=O)NO ; CC(C)C(NS(=O)(=O)c1ccc(C(C)(C)C)cc1)C(=O)NO ; Cc1cc(C)c(S(=O)(=O)NC(C(=O)NO)C(C)C)c(C)c1 ; O=C(NO)C1CCCCN1S(=O)(=O)c1ccc(F)cc1 ; O=C(NO)C1CCCCN1S(=O)(=O)c1ccc(C23CC4CC(CC(C4)C2)C3)cc1]

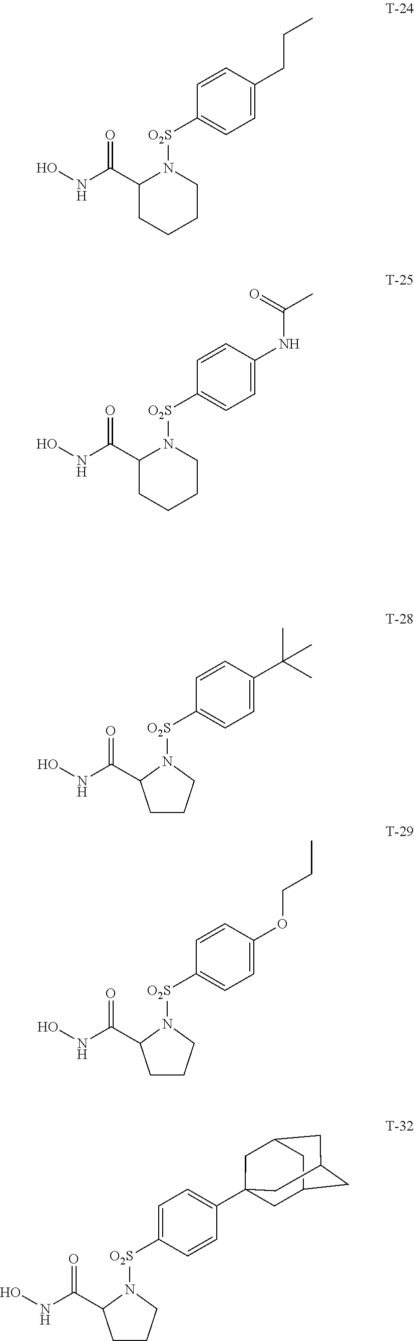 [SMILES: CCCc1ccc(S(=O)(=O)N2CCCCC2C(=O)NO)cc1 ; CC(=O)Nc1ccc(S(=O)(=O)N2CCCCC2C(=O)NO)cc1 ; CC(C)(C)c1ccc(S(=O)(=O)N2CCCC2C(=O)NO)cc1 ; CCCOc1ccc(S(=O)(=O)N2CCCC2C(=O)NO)cc1 ; O=C(NO)C1CCCN1S(=O)(=O)c1ccc(C23CC4CC(CC(C4)C2)C3)cc1]

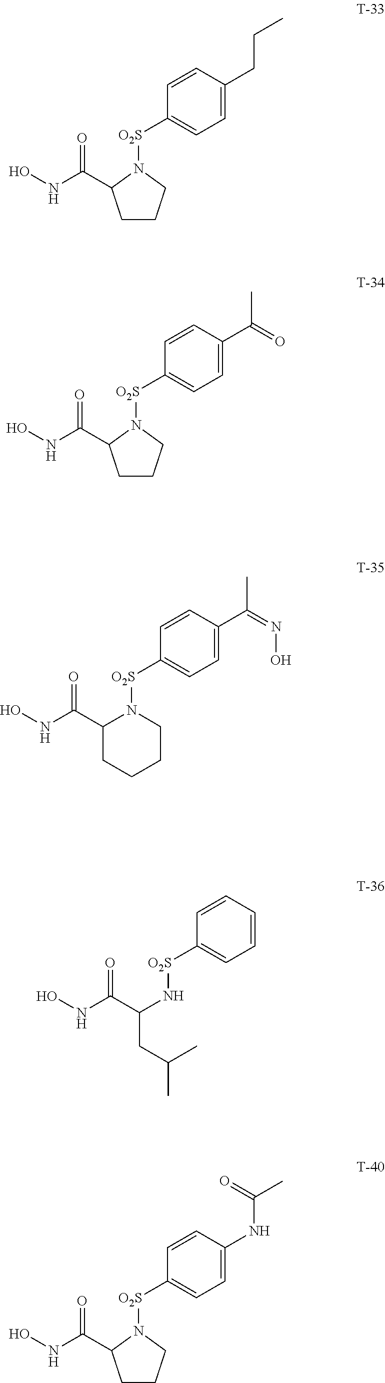 [SMILES: CCCc1ccc(S(=O)(=O)N2CCCC2C(=O)NO)cc1 ; CC(=O)c1ccc(S(=O)(=O)N2CCCC2C(=O)NO)cc1 ; C/C(=N/O)c1ccc(S(=O)(=O)N2CCCCC2C(=O)NO)cc1 ; CC(C)CC(NS(=O)(=O)c1ccccc1)C(=O)NO ; CC(=O)Nc1ccc(S(=O)(=O)N2CCCC2C(=O)NO)cc1]

In an embodiment, the compound represented by Chemical Formula 1 may be any one selected from the following compounds.
N-hydroxy-1-(phenylsulfonyl) piperidine-2-carboxamide,
1-([1,1′-biphenyl]-4-ylsulfonyl)-N-hydroxypiperidine-2-carboxamide,
1-((4-(tert-butyl)phenyl) sulfonyl)-N-hydroxypiperidine-2-carboxamide,
2-([1,1′-biphenyl]-4-sulfonamido)-N-hydroxy-3-phenylpropanamide,
2-((4-(tert-butyl)phenyl) sulfonamido)-N-hydroxy-4-methylpentanamide,
2-((4-butoxyphenyl) sulfonamido)-N-hydroxy-4-methylpentanamide,
N-hydroxy-4-methyl-2-((2,4,6-trimethylphenyl) sulfonamido) pentanamide,
2-([1,1′-biphenyl]-4-sulfonamido)-N-hydroxy-3-methylbutanamide,
1-((4-((3r,5r, 7r)-adamantan-1-yl)phenyl) sulfonyl)-N-hydroxypiperidine-2-carboxamide,
1-((4-butoxyphenyl) sulfonyl)-N-hydroxypyrrolidine-2-carboxamide, and
N-hydroxy-1-((4-propylphenyl) sulfonyl) pyrrolidine-2-carboxamide.

In an embodiment, the compound represented by Chemical Formula 1 may be derived from an amino acid.

For example, the compound represented by Chemical Formula 1 may be derived from any one of the following amino acids.

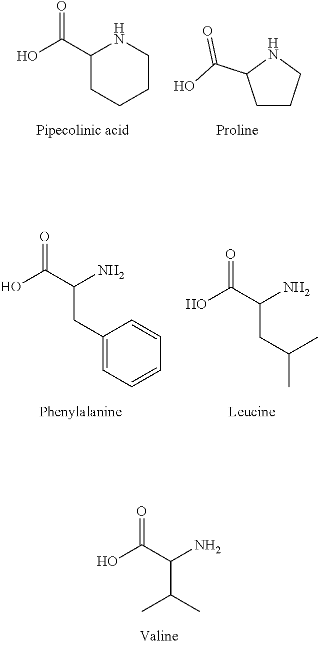

In an embodiment, the composition may be for activating the phagocytosis of macrophages.

In an embodiment, the composition may be for activating the phagocytosis of macrophages against Staphylococcus aureus (S. aureus).

In an embodiment, the composition may be for strengthening the skin barrier.

In an embodiment, the composition may be for suppressing skin inflammation.

In an embodiment, the composition may be a cosmetic composition.

In an embodiment, the external form of the cosmetic composition may contain a cosmetically or dermatologically acceptable medium or base. It may be provided in any formulation suitable for topical application, for example, in the form of a solution, a gel, a solid, a pasty anhydrous product, an emulsion obtained by dispersing an oil phase in an aqueous phase, a suspension, a microemulsion, a microcapsule, a microgranule or an ionic (liposome) and non-ionic vesicular dispersing agent, or in the form of a cream, a skin toner, a lotion, a powder, an ointment, a spray, or a conceal stick. These compositions may be prepared by a typical method in the field.

Furthermore, the composition according to the present disclosure may also be used in the form of a foam or in the form of an aerosol composition that further contains a compressed propellant.

The cosmetic composition according to the present disclosure is not particularly limited in its formulation, and may be formulated into a cosmetic such as, for example, a softening toner, an astringent toner, a nourishing toner, a nourishing cream, a massage cream, an essence, an eye cream, an eye essence, a cleansing cream, a cleansing foam, a cleansing water, a cleansing tissue including the cosmetic composition, a pack, a powder, a body lotion, a body cream, a body oil, and a body essence.

When the formulation of the cosmetic composition according to the present disclosure is a paste, a cream or a gel, animal fiber, plant fiber, wax, paraffin, starch, tragacanth, a cellulose derivative, polyethylene glycol, silicone, bentonite, silica, talc, zinc oxide, or the like may be used as a carrier ingredient.

When the formulation of the cosmetic composition according to the present disclosure is a powder or a spray, lactose, talc, silica, aluminum hydroxide, calcium silicate, or a polyamide powder may be used as the carrier ingredient, and in particular, when the formulation of the cosmetic composition of the present disclosure is a spray, the formulation may additionally include a propellant such as a chlorofluorohydrocarbon, propane/butane or dimethyl ether.

When the formulation of the cosmetic composition according to the present disclosure is a solution or an emulsion, a solvent, a solubilizer or an emulsifier is used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan.

When the formulation of the cosmetic composition of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier ingredient.

The cosmetic composition according to the present disclosure may further include functional additives and ingredients included in general cosmetic compositions. The functional additive may include an ingredient selected from the group consisting of a water-soluble vitamin, an oil-soluble vitamin, a polymeric peptide, a polymeric polysaccharide, squalane, a sphingolipid, and a seaweed extract.

The cosmetic composition according to the present disclosure may also be blended with ingredients contained in general cosmetic compositions, if necessary, in addition to the functional additives. Examples of other blended ingredients which may be contained include oil and fat ingredients, a moisturizer, an emollient, an emulsifier, organic and inorganic pigments, an organic powder, an ultraviolet absorbent, a preservative, a disinfectant, an antioxidant, a plant extract, a pH adjuster, an alcohol, a colorant, a fragrance, a circulation promoter, a cooling agent, an antiperspirant, purified water, and the like.

In an embodiment, the composition may be a skin external preparation.

The skin external preparation is a general term that may include anything that is applied externally to the skin, and may include cosmetics, medicines, and the like in various formulations.

In an embodiment, the composition may be a food composition.

In an embodiment, the composition may be an oral composition or a non-therapeutic oral composition.

The food composition or oral composition according to the present disclosure may be a liquid or solid formulation, and may be a tablet, a capsule, a soft capsule, a pill, a granule, a beverage (drink), a diet bar, chocolate, a caramel formulation, or a confectionery formulation, and the formulation is not particularly limited.

The food composition or oral composition of the present disclosure may contain, in addition to the active ingredient, excipients, sugars, fragrances, colorants, oils and fats, proteins, and the like, if necessary.

In an embodiment, the composition may be a pharmaceutical composition.

The pharmaceutical composition may further contain a pharmaceutical adjuvant such as a preservative, stabilizer, hydrating agent, or an emulsion-promoting agent, a salt and/or buffer for adjusting osmotic pressure, and other therapeutically useful substances, and may be formulated into various formulations for oral or parenteral administration by typical methods.

Examples of the formulation for oral administration include a tablet, a pill, a hard/soft capsule, a solution, a suspension, an emulsifier, a syrup, a powder, a dust, a fine granule, a granule, a pellet, and the like, and these formulations contain a surfactant, a diluent (for example: lactose, dextrose, sucrose, mannitol, sorbitol, cellulose and glycine) and a lubricant (for example: silica, talc, stearic acid, and a magnesium or calcium salt thereof, and polyethylene glycol) in addition to the active ingredient. The tablet may also contain a binder such as magnesium aluminum silicate, starch paste, gelatin, tragacanth, methyl cellulose, sodium carboxymethyl cellulose, and polyvinylpyrrolidine, and may contain a pharmaceutical additive such as a disintegrant such as starch, agar, alginic acid, or a sodium salt thereof, an absorbent, a colorant, a flavoring agent, a sweetener, and the like in some cases. The tablet may be prepared by a typical mixing, granulation or coating method. Further, the parenteral administration form may be a transdermal administration type formulation, and may be a formulation such as, for example, an injection, a drop, an ointment, a lotion, a gel, a cream, a spray, a suspension, an emulsion, a suppository, or a patch, but is not limited thereto.

The pharmaceutical composition may be administered parenterally, rectally, topically, transdermally, subcutaneously, and the like.

In an embodiment, the pharmaceutical composition may be for preventing, ameliorating, or treating skin inflammation.

In an embodiment, the pharmaceutical composition may be for treating a subject with impaired phagocytosis of macrophages.

In an embodiment, the pharmaceutical composition may be for treating a subject with impaired phagocytosis of macrophages against S. aureus.

As used herein, the term “prevention” refers to all actions that suppress a disease or delay the onset of the disease by administering the pharmaceutical composition according to the present disclosure.

As used herein, term “amelioration” refers to all actions that at least reduce a parameter associated with a condition to be treated, for example, the degree of symptoms.

As used herein, the term “treatment” refers to all actions that ameliorate or beneficially change symptoms caused by a disease by administering the pharmaceutical composition according to the present disclosure.

In an embodiment, the content of the active ingredient may be 0.01 μM to 100 mM based on the total volume of the composition.

For example, the content may be 0.01 μM or more, 0.05 UM or more, 0.1 μM or more, 1 μM or more, 10 μM or more, 100 UM or more, 0.5 mM or more, 1 mM or more, 1.5 mM or more, 2 mM or more, 2.5 mM or more, 10 mM or more, or 50 mM or more, and 100 mM or less, 50 mM or less, 10 mM or less, 8 mM or less, 6 mM or less, 4 mM or less, 1 mM or less, 100 μM or less, 10 μM or less, 1 μM or less, 0.1 μM or less, or 0.05 μM or less.

For example, the content may be 0.01 μM to 100 mM, 0.05 μM to 10 mM, or 0.1 μM to 4 mM based on the total volume of the composition.

When the concentration is less than 0.01 μM, the effect may be negligible, and when the concentration exceeds 100 mM, a cytotoxicity problem may occur due to the high concentration.

In an embodiment, the daily application amount of active ingredient may be 1 to 1,000 mg/kg.

For example, the daily application amount may be a daily dosage, or may refer to a daily intake for the human body.

For example, the daily application amount may be 1 mg/kg or more, 10 mg/kg or more, 100 mg/kg or more, 300 mg/kg or more, 500 mg/kg or more, 700 mg/kg or more, or 900 mg/kg or more, and 1,000 mg/kg or less, 800 mg/kg or less, 600 mg/kg or less, 400 mg/kg or less, 200 mg/kg or less, 100 mg/kg or less, or 10 mg/kg or less.

In an embodiment, cells may be treated with the active ingredient at a concentration of 1 to 100 μM.

For example, cells may be treated with the active ingredient at a concentration of 1 μM or more, 10 μM or more, 30 μM or more, 50 μM or more, 70 μM or more, or 90 μM or more, and at a concentration of 100 μM or less, 80 μM or less, 60 μM or less, 40 μM or less, or 20 μM or less.

Hereinafter, the present disclosure will be described in more detail through the Examples. However, the following Examples are provided only for illustrative purposes to facilitate the understanding of the present disclosure, and the purview and scope of the present disclosure is not limited thereto.

### EXAMPLES

### Preparation Examples

Novel compounds were prepared as follows, and the IUPAC names of the novel compounds are shown in the following Table 1.

TABLE 1

| TM No. | Compound Name |
|---|---|
| T-01 | N-hydroxy-1-(phenylsulfonyl)piperidine-2-carboxamide |
| T-02 | 1-([1,1′-biphenyl]-4-ylsulfonyl)-N-hydroxypiperidine-2-carboxamide |
| T-03 | 1-((4-(tert-butyl)phenyl)sulfonyl)-N-hydroxypiperidine-2-carboxamide |
| T-04 | 1-((4-butoxyphenyl)sulfonyl)-N-hydroxypiperidine-2-carboxamide |
| T-05 | N-hydroxy-1-(mesitylsulfonyl)piperidine-2-carboxamide |
| T-07 | 2-([1,1′-biphenyl]-4-sulfonamido)-N-hydroxy-3-phenylpropanamide |
| T-08 | 2-((4-(tert-butyl)phenyl)sulfonamido)-N-hydroxy-3-phenylpropanamide |
| T-10 | N-hydroxy-3-phenyl-2-((2,4,6-trimethylphenyl)sulfonamido)propanamide |
| T-11 | N-hydroxy-2-(naphthalene-2-sulfonamido)-3-phenylpropanamide |
| T-12 | 2-([1,1′-biphenyl]-4-sulfonamido)-N-hydroxy-4-methylpentanamide |
| T-13 | 2-((4-(tert-butyl)phenyl)sulfonamido)-N-hydroxy-4-methylpentanamide |
| T-14 | 2-((4-butoxyphenyl)sulfonamido)-N-hydroxy-4-methylpentanamide |
| T-15 | N-hydroxy-4-methyl-2-((2,4,6-trimethylphenyl)sulfonamido)pentanamide |
| T-17 | 2-([1,1′-biphenyl]-4-sulfonamido)-N-hydroxy-3-methylbutanamide |
| T-18 | 2-((4-(tert-butyl)phenyl)sulfonamido)-N-hydroxy-3-methylbutanamide |
| T-20 | N-hydroxy-3-methyl-2-((2,4,6-trimethylphenyl)sulfonamido)butanamide |
| T-21 | 1-((4-fluorophenyl)sulfonyl)-N-hydroxypiperidine-2-carboxamide |
| T-23 | 1-((4-((3r,5r,7r)-adamantan-1-yl)phenyl)sulfonyl)-N-hydroxypiperidine-2-carboxamide |
| T-24 | N-hydroxy-1-((4-propylphenyl)sulfonyl)piperidine-2-carboxamide |
| T-25 | 1-((4-acetamidophenyl)sulfonyl)-N-hydroxypiperidine-2-carboxamide |
| T-28 | 1-((4-(tert-butyl)phenyl)sulfonyl)-N-hydroxypyrrolidine-2-carboxamide |
| T-29 | 1-((4-butoxyphenyl)sulfonyl)-N-hydroxypyrrolidine-2-carboxamide |
| T-32 | 1-((4-((3r,5r,7r)-adamantan-1-yl)phenyl)sulfonyl)-N-hydroxypyrrolidine-2-carboxamide |
| T-33 | N-hydroxy-1-((4-propylphenyl)sulfonyl)pyrrolidine-2-carboxamide |
| T-34 | 1-((4-acetylphenyl)sulfonyl)-N-hydroxypyrrolidine-2-carboxamide |
| T-35 | N-hydroxy-1-((4-(1-(hydroxyimino)ethyl)phenyl)sulfonyl)piperidine-2-carboxamide |
| T-36 | N-hydroxy-4-methyl-2-(phenylsulfonamido)pentanamide |
| T-40 | 1-((4-acetamidophenyl)sulfonyl)-N-hydroxypyrrolidine-2-carboxamide |

### (1) Synthesis of T-01 N-hydroxy-1-(phenylsulfonyl) piperidine-2-carboxamide

After 1.69 g of pipecolinic acid was well stirred together with 30 mL of THE, 30 mL of 1 M Na₂CO₃ was added. 2 mL of benzenesulfonyl chloride was slowly added dropwise at 0° C., and the resulting mixture was stirred at the same temperature for 1 hour. After washing with 30 mL of diethyl ether, the pH of the aqueous layer was adjusted to pH 3 with 3 M HCl. After extraction with ethyl acetate, the resulting product was dried using MgSO₄ and then concentrated under reduced pressure to obtain 1.89 g of an intermediate 1-(phenylsulfonyl) piperidine-2-carboxylic acid. The intermediate was used for the next reaction without a further purification process.

After 1.89 g of 1-(phenylsulfonyl) piperidine-2-carboxylic acid was dissolved in 15 mL of THF together with 0.85 mL of N-methylmorpholine-N-oxide (NMO), the resulting solution was stirred at 0° C. After 0.73 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.73 g of hydroxylamine hydrochloride and 1.46 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:2) to obtain 0.72 g of N-hydroxy-1-(phenylsulfonyl) piperidine-2-carboxamide.

¹H-NMR (500 MHz, DMSO-d₆): δ10.62 (s, 1H), 8.79 (s, 1H), 7.78 (m, 2H), 7.66 (m, 1H), 7.57 (m, 2H), 4.34 (m, 1H), 3.63 (m, 1H), 3.47 (m, 1H), 1.78 (m, 1H), 1.55 (m, 1H) 1.45-1.40 (m, 3H), 1.17 (m, 1H)

### (2) Synthesis of T-02 1-([1,1′-biphenyl]-4-ylsulfonyl)-N-hydroxypiperidine-2-carboxamide

After 0.65 g of pipecolinic acid was well stirred together with 20 mL of THF, 11.5 mL of 1 M Na₂CO₃ was added. 1.52 g of 4-biphenylsulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with ethyl acetate, the resulting product was dried using MgSO₄ and then concentrated under reduced pressure to obtain 0.78 g of an intermediate 1-([1,1′-biphenyl]-4-ylsulfonyl) piperidine-2-carboxylic acid. The intermediate was used for the next reaction without a further purification process.

After 0.7 g of 1-([1,1′-biphenyl]-4-ylsulfonyl) piperidine-2-carboxylic acid was dissolved in 20 mL of THF together with 0.25 mL of N-methylmorpholine-N-oxide (NMO), the resulting solution was stirred at 0° C. After 0.21 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.21 g of hydroxylamine hydrochloride and 0.42 mL of triethylamine dissolved in 10 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:2) to obtain 0.26 g of 1-([1,1′-biphenyl]-4-ylsulfonyl)-N-hydroxypiperidine-2-carboxamide.

¹H-NMR (500 MHz, DMSO-d₆): δ10.67 (s, 1H), 8.80 (s, 1H), 7.87 (m, 4H), 7.77 (m, 2H), 7.52 (m, 2H), 7.45 (m, 1H), 4.38 (m, 1H), 3.66 (m, 1H), 3.50 (m, 1H), 1.81 (m, 1H), 1.59 (m, 1H), 1.47-1.43 (m, 3H), 1.26-1.15 (m, 1H)

### (3) Synthesis of T-03 1-((4-(tert-butyl)phenyl) sulfonyl)-N-hydroxypiperidine-2-carboxamide

After 0.65 g of pipecolinic acid was well stirred together with 20 mL of THF, 11.5 mL of 1 M Na₂CO₃ was added. 1.4 g of 4-tert-butylbenzenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with ethyl acetate, the resulting product was dried using MgSO₄ and then concentrated under reduced pressure to obtain 0.78 g of an intermediate 1-((4-(tert-butyl)phenyl) sulfonyl) piperidine-2-carboxylic acid. The intermediate was used for the next reaction without a further purification process.

After 0.8 g of 1-((4-(tert-butyl)phenyl) sulfonyl) piperidine-2-carboxylic acid was dissolved in 30 mL of THF together with 0.29 mL of N-methylmorpholine-N-oxide (NMO), the resulting solution was stirred at 0° C. After 0.25 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.25 g of hydroxylamine hydrochloride and 0.5 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.41 g of 1-((4-(tert-butyl)phenyl) sulfonyl)-N-hydroxypiperidine-2-carboxamide.

¹H-NMR (500 MHz, DMSO-d₆): δ10.62 (s, 1H), 8.81 (s, 1H), 7.69 (m, 2H), 7.59 (m, 2H), 4.34 (m, 1H), 3.59 (m, 1H), 3.43 (m, 1H), 1.78 (m, 1H), 1.54 (m, 1H), 1.44 (m, 3H), 1.31 (s, 9H), 1.16 (m, 1H)

### (4) Synthesis of T-04 1-((4-butoxyphenyl) sulfonyl)-N-hydroxypiperidine-2-carboxamide

After 0.65 g of pipecolinic acid was well stirred together with 20 mL of THF, 11.5 mL of 1 M Na₂CO₃ was added. 1.2 mL of 4-butoxybenzene-1-sulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 1.17 g of an intermediate 1-((4-butoxyphenyl) sulfonyl) piperidine-2-carboxylic acid. The intermediate was used for the next reaction without a further purification process.

After 1.17 g of 1-((4-butoxyphenyl) sulfonyl) piperidine-2-carboxylic acid was dissolved in 30 mL of THF together with 0.42 mL of N-methylmorpholine-N-oxide (NMO), the resulting solution was stirred at 0° C. After 0.36 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.36 g of hydroxylamine hydrochloride and 0.72 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.37 g of 1-((4-butoxyphenyl) sulfonyl)-N-hydroxypiperidine-2-carboxamide.

¹H-NMR (500 MHz, DMSO-d₆): δ10.61 (s, 1H), 8.77 (s, 1H), 7.67 (d, 2H, J=8.5 Hz), 7.06 (d, 2H, J=9 Hz), 4.30 (m, 1H), 4.05 (t, 2H, J=7 Hz), 3.59 (m, 1H), 3.44 (m, 1H) 1.77-1.69 (m, 3H), 1.54-1.42 (m, 6H), 1.18 (m, 1H), 0.94 (t, 3H, J=7.5 Hz)

### (5) Synthesis of T-05 N-hydroxy-1-(mesitylsulfonyl) piperidine-2-carboxamide

After 0.65 g of pipecolinic acid was well stirred together with 20 mL of THF, 11.5 mL of 01 M Na₂CO₃ was added. 1.31 g of mesitylenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 0.71 g of an intermediate 1-(mesitylsulfonyl) piperidine-2-carboxylic acid. The intermediate was used for the next reaction without a further purification process.

After 0.69 g of 1-(mesitylsulfonyl) piperidine-2-carboxylic acid was dissolved in 20 mL of THF together with 0.27 mL of N-methylmorpholine-N-oxide (NMO), the resulting solution was stirred at 0° C. After 0.23 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.23 g of hydroxylamine hydrochloride and 0.46 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.14 g of N-hydroxy-1-(mesitylsulfonyl) piperidine-2-carboxamide.

¹H-NMR (500 MHz, DMSO-d₆): δ10.48 (s, 1H), 8.86 (s, 1H), 7.05 (s, 2H), 3.69 (t, 1H, J=10.5 Hz), 3.32 (s, 3H), 3.43 (s, 3H) 1.79 (m, 2H), 1.65-1.50 (m, 6H), 1.30-1.23 (m, 3H)

### (6) Synthesis of T-07 2-([1,1′-biphenyl]-4-sulfonamido)-N-hydroxy-3-phenylpropanamide

After 0.83 g of phenylalanine was well stirred together with 20 mL of THE, 11.5 mL of 1 M Na₂CO₃ was added. 1.52 g of 4-biphenylsulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 1.26 g of an intermediate ([1,1′-biphenyl]-4-ylsulfonyl)phenylalanine. The intermediate was used for the next reaction without a further purification process.

After 1.2 g of 1-([1,1′-biphenyl]-4-ylsulfonyl)phenylalanine was dissolved in 20 mL of THF together with 0.38 mL of N-methylmorpholine-N-oxide (NMO), the resulting solution was stirred at 0° C. After 0.33 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.33 g of hydroxylamine hydrochloride and 0.66 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=2:3) to obtain 0.32 g of 2-([1,1′-biphenyl]-4-sulfonamido)-N-hydroxy-3-phenylpropanamide.

¹H-NMR (500 MHz, DMSO-d₆): δ10.07 (s, 1H), 8.88 (s, 1H), 8.27 (d, 2H, J=9.0 Hz), 7.69 (m, 3H), 7.59 (m, 2H), 7.53 (m, 2H), 7.43 (m, 1H), 7.16 (m, 3H), 7.07 (m, 2H), 3.82 (m, 1H), 2.80 (m, 1H), 2.63 (m, 1H)

### (7) Synthesis of T-08 2-((4-(tert-butyl)phenyl) sulfonamido)-N-hydroxy-3-phenylpropanamide

After 0.83 g of phenylalanine was well stirred together with 20 mL of THF, 11.5 mL of 1 M Na₂CO₃ was added. 1.4 g of 4-tert-butylbenzenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 1.31 g of an intermediate ((4-(tert-butyl)phenyl) sulfonyl)phenylalanine. The intermediate was used for the next reaction without a further purification process.

After 1.2 g of ((4-(tert-butyl)phenyl) sulfonyl)phenylalanine was dissolved in 20 mL of THF together with 0.4 mL of N-methylmorpholine-N-oxide (NMO), the resulting solution was stirred at 0° C. After 0.35 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.35 g of hydroxylamine hydrochloride and 0.69 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.38 g of 2-((4-(tert-butyl)phenyl) sulfonamido)-N-hydroxy-3-phenylpropanamide.

¹H-NMR (500 MHz, DMSO-d₆): δ10.61 (s, 1H), 8.88 (s, 1H), 8.13 (d, 1H, J=8.5 Hz), 7.40 (m, 4H), 7.12 (m, 3H), 7.00 (m, 2H), 3.71 (m, 1H), 2.76 (m, 1H), 2.55 (m, 1H), 1.26 (s, 9H)

### (8) Synthesis of T-10 N-hydroxy-3-phenyl-2-((2,4,6-trimethylphenyl) sulfonamido) propanamide

After 0.83 g of phenylalanine was well stirred together with 20 mL of THE, 11.5 mL of 1 M Na₂CO₃ was added. 1.31 g of mesitylenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 1 g of an intermediate (mesitylsulfonyl)phenylalanine. The intermediate was used for the next reaction without a further purification process.

After 1 g of (mesitylsulfonyl)phenylalanine was dissolved in 20 mL of THF together with 0.35 mL of N-methylmorpholine-N-oxide (NMO), the resulting solution was stirred at 0° C. After 0.3 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.3 g of hydroxylamine hydrochloride and 0.6 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.25 g of N-hydroxy-3-phenyl-2-((2,4,6-trimethylphenyl) sulfonamido) propanamide.

¹H-NMR (500 MHz, DMSO-d₆): δ10.59 (s, 1H), 8.87 (s, 1H), 7.89 (d, 1H, J=9.5 Hz), 7.07 (m, 3H), 6.95 (m, 2H), 6.82 (m, 2H), 3.67 (m, 1H), 2.76 (m, 1H), 2.60 (m, 1H), 2.49 (s, 6H), 2.26 (s, 3H)

### (9) Synthesis of T-11 N-hydroxy-2-(naphthalene-2-sulfonamido)-3-phenylpropanamide

After 0.83 g of phenylalanine was well stirred together with 20 mL of THE, 11.5 mL of 1 M Na₂CO₃ was added. 1.36 g of 2-naphthalenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 1.2 g of an intermediate (naphthalen-2-ylsulfonyl)phenylalanine. The intermediate was used for the next reaction without a further purification process.

After 1.1 g of (naphthalen-2-ylsulfonyl)phenylalanine was dissolved in 20 mL of THF together with 0.37 mL of N-methylmorpholine-N-oxide (NMO), the resulting solution was stirred at 0° C. After 0.32 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.32 g of hydroxylamine hydrochloride and 0.65 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:2) to obtain 0.21 g of N-hydroxy-2-(naphthalene-2-sulfonamido)-3-phenylpropanamide.

¹H-NMR (500 MHz, DMSO-d₆): δ10.63 (s, 1H), 8.84 (s, 1H), 8.32 (d, 1H, J=9.5 Hz), 8.19 (s, 1H), 8.19 (m, 2H), 7.92 (d, 1H, J=9.5 Hz), 7.65 (m, 2H), 7.56 (m, 1H), 7.28-7.00 (m, 5H), 3.84 (q, 1H), 2.78 (m, 1H), 2.59 (m, 1H)

### (10) Synthesis of T-12 2-([1,1′-biphenyl]-4-sulfonamido)-N-hydroxy-4-methylpentanamide

After 0.66 g of leucine was well stirred together with 20 mL of THE, 11.5 mL of 1 M Na₂CO₃ was added. 1.52 g of 4-biphenylsulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 0.64 g of an intermediate ([1,1′-biphenyl]-4-ylsulfonyl) leucine. The intermediate was used for the next reaction without a further purification process.

After 0.6 g of ([1,1′-biphenyl]-4-ylsulfonyl) leucine was dissolved in 20 mL of THF together with 0.21 mL of N-methylmorpholine-N-oxide (NMO), the resulting solution was stirred at 0° C. After 0.18 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.18 g of hydroxylamine hydrochloride and 0.36 mL of triethylamine dissolved in 10 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.34 g of 2-([1,1′-biphenyl]-4-sulfonamido)-N-hydroxy-4-methylpentanamide.

¹H-NMR (500 MHz, DMSO-d₆): δ10.66 (s, 1H), 8.81 (s, 1H), 8.03 (d, 1H, J=6.0 Hz), 7.83 (m, 4H), 7.73 (d, 2H, J=7.0 Hz), 7.51 (m, 2H), 7.43 (m, 1H), 3.59 (q, 1H), 1.41 (m, 1H), 1.33-1.21 (m, 2H), 0.75 (d, 3H, J=6.5 Hz), 0.64 (d, 3H, J=7.0 Hz)

### (11) Synthesis of T-13 2-((4-(tert-butyl)phenyl) sulfonamido)-N-hydroxy-4-methylpentanamide

After 0.66 g of leucine was well stirred together with 20 mL of THE, 11.5 mL of 1 M Na₂CO₃ was added. 1.4 g of 4-tert-butylbenzenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 1.09 g of an intermediate ((4-(tert-butyl)phenyl) sulfonyl) leucine. The intermediate was used for the next reaction without a further purification process.

After 1.07 g of ((4-(tert-butyl)phenyl) sulfonyl) leucine was dissolved in 20 mL of THF together with 0.4 mL of N-methylmorpholine-N-oxide (NMO), the resulting solution was stirred at 0° C. After 0.35 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.35 g of hydroxylamine hydrochloride and 0.7 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.15 g of 2-((4-(tert-butyl)phenyl) sulfonamido)-N-hydroxy-4-methylpentanamide.

¹H-NMR (500 MHz, DMSO-d₆): δ10.64 (s, 1H), 8.83 (s, 1H), 7.88 (m, 1H), 7.70 (d, 2H, J=9.0 Hz), 7.57 (d, 2H, J=8.5 Hz), 3.49 (m, 1H), 1.32-1.24 (m, 11H), 1.12 (m, 1H), 0.69 (d, 3H, J=6.5 Hz), 0.55 (d, 3H, J=6.5 Hz)

### (12) Synthesis of T-14 2-((4-butoxyphenyl) sulfonamido)-N-hydroxy-4-methylpentanamide

After 0.66 g of leucine was well stirred together with 20 mL of THF, 11.5 mL of 1 M Na₂CO₃ was added. 1.2 mL of 4-butoxybenzene-1-sulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 1 g of an intermediate ((4-butoxyphenyl) sulfonyl) leucine. The intermediate was used for the next reaction without a further purification process.

After 1 g of ((4-butoxyphenyl) sulfonyl) leucine was dissolved in 20 mL of THF together with 0.35 mL of N-methylmorpholine-N-oxide (NMO), the resulting solution was stirred at 0° C. After 0.3 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.3 g of hydroxylamine hydrochloride and 0.6 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 40 mg of 2-((4-butoxyphenyl) sulfonamido)-N-hydroxy-4-methylpentanamide.

¹H-NMR (500 MHz, DMSO-d₆): δ10.61 (s, 1H), 8.77 (s, 1H), 7.76 (m, 1H), 7.67 (d, 2H, J=9.0 Hz), 7.04 (d, 2H, J=8.5 Hz), 4.04 (t, 2H, J=6.5 Hz), 3.50 (m, 1H), 1.70 (m, 2H), 1.45 (m, 3H), 1.27 (m, 1H), 1.19 (m, 1H), 0.93 (t, 3H, J=7.0 Hz), 0.77-0.72 (d, 3H, J=6.5 Hz), 0.63 (d, 3H, J=6.0 Hz)

### (13) Synthesis of T-15 N-hydroxy-4-methyl-2-((2,4,6-trimethylphenyl) sulfonamido) pentanamide

After 0.66 g of leucine was well stirred together with 20 mL of THF, 11.5 mL of 1 M Na₂CO₃ was added. 1.31 g of 2-mesitylenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 0.76 g of an intermediate (mesitylsulfonyl) leucine. The intermediate was used for the next reaction without a further purification process.

After 0.75 g of (mesitylsulfonyl) leucine was dissolved in 20 mL of THF together with 0.29 mL of N-methylmorpholine-N-oxide (NMO), the resulting solution was stirred at 0° C. After 0.25 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.25 g of hydroxylamine hydrochloride and 0.5 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.14 g of N-hydroxy-4-methyl-2-((2,4,6-trimethylphenyl) sulfonamido) pentanamide.

¹H-NMR (500 MHz, DMSO-d₆): δ10.51 (s, 1H), 8.88 (s, 1H), 7.77 (m, 1H), 6.98 (s, 2H), 3.45 (m, 1H), 2.55 (s, 3H), 2.50 (s, 3H), 2.24 (s, 3H), 1.41 (m, 1H), 1.33-1.20 (m, 2H), 0.73 (d, 3H, J=6.5 Hz), 0.55 (d, 3H, J=6.5 Hz)

### (14) Synthesis of T-17 2-([1,1′-biphenyl]-4-sulfonamido)-N-hydroxy-3-methylbutanamide

After 0.59 g of valine was well stirred together with 20 mL of THE, 11.5 mL of 1 M Na₂CO₃ was added. 1.52 g of 4-biphenylsulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 0.48 g of an intermediate ([1,1′-biphenyl]-4-ylsulfonyl) valine. The intermediate was used for the next reaction without a further purification process.

After 0.48 g of ([1,1′-biphenyl]-4-ylsulfonyl) valine was dissolved in 20 mL of THF together with 0.17 mL of N-methylmorpholine-N-oxide (NMO), the resulting solution was stirred at 0° C. After 0.15 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.15 g of hydroxylamine hydrochloride and 0.3 mL of triethylamine dissolved in 10 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.1 g of 2-([1,1′-biphenyl]-4-sulfonamido)-N-hydroxy-4-methylbutanamide.

¹H-NMR (500 MHz, DMSO-d₆): δ10.56 (s, 1H), 8.79 (s, 1H), 8.00 (s, 1H), 7.83 (m, 4H), 7.73 (d, 2H, J=7.0 Hz), 7.51 (m, 2H), 7.43 (m, 1H), 3.33 (m, 1H), 1.77 (m, 1H), 0.77 (d, 3H, J=6.5 Hz), 0.75 (d, 3H, J=5.5 Hz)

### (15) Synthesis of T-18 2-((4-(tert-butyl)phenyl) sulfonamido)-N-hydroxy-3-methylbutanamide

After 0.59 g of valine was well stirred together with 20 mL of THE, 11.5 mL of 1 M Na₂CO₃ was added. 1.4 g of 4-tert-butylbenzenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 0.71 g of an intermediate ((4-(tert-butyl)phenyl) sulfonyl) valine. The intermediate was used for the next reaction without a further purification process.

After 0.7 g of ((4-(tert-butyl)phenyl) sulfonyl) valine was dissolved in 20 mL of THF together with 0.25 mL of N-methylmorpholine-N-oxide (NMO), the resulting solution was stirred at 0° C. After 0.21 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.21 g of hydroxylamine hydrochloride and 0.42 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.3 g of 2-((4-(tert-butyl)phenyl) sulfonamido)-N-hydroxy-3-methylbutanamide.

¹H-NMR (500 MHz, DMSO-d₆): δ10.50 (s, 1H), 8.80 (s, 1H), 7.81 (m, 1H), 7.68 (d, 2H, J=8.0 Hz), 7.54 (d, 2H, J=8.0 Hz), 3.27 (m, 1H), 1.75 (m, 1H), 1.29 (s, 9H), 0.73 (d, 3H, J=6.5 Hz), 0.70 (d, 3H, J=6.5 Hz)

### (16) Synthesis of T-20 N-hydroxy-3-methyl-2-((2,4,6-trimethylphenyl) sulfonamido) butanamide

After 0.59 g of valine was well stirred together with 20 mL of THE, 11.5 mL of 1 M Na₂CO₃ was added. 1.31 g of 2-mesitylenesulfonyl chloride was slowly added dropwise at 0° C., and the resulting mixture was stirred at room temperature for 1 hour. After washing twice with 50 mL of diethyl ether, the pH of the aqueous layer was adjusted to pH 3 with 3 M HCl. After extraction with 100 mL of ethyl acetate, the resulting product was dried using MgSO₄ and then concentrated under reduced pressure to obtain 0.53 g of an intermediate (mesitylsulfonyl) valine. The intermediate was used for the next reaction without a further purification process.

After 0.52 g of (mesitylsulfonyl) valine was dissolved in 20 mL of THF together with 0.25 mL of N-methylmorpholine-N-oxide (NMO), the resulting solution was stirred at 0° C. After 0.21 mL of ethyl chloroformate was slowly added dropwise, the resulting mixture was stirred at the same temperature for 30 minutes. A filtrate obtained after filtering out a solid from the reaction solution was separately stored, and then added dropwise to a solution of 0.21 g of hydroxylamine hydrochloride and 0.42 mL of triethylamine dissolved in 20 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.14 g of N-hydroxy-3-methyl-2-((2,4,6-trimethylphenyl) sulfonamido) butanamide.

¹H-NMR (500 MHz, DMSO-d₆): δ10.43 (s, 1H), 8.80 (s, 1H), 7.59 (d, 1H, J=9.5 Hz), 6.98 (s, 2H), 3.18 (t, 1H, J=8.0 Hz), 2.59-2.50 (m, 6H), 2.24 (s, 3H), 1.76 (m, 1H), 0.70 (m, 6H)

### (17) Synthesis of T-21 1-((4-fluorophenyl) sulfonyl)-N-hydroxypiperidine-2-carboxamide

After 500 mg of pipecolinic acid was well stirred together with 8 mL of THE, 9.6 mL of 1 M Na₂CO₃ was added. 904 mg of 4-fluorobenzenesulfonyl chloride was slowly added dropwise at 0° C., and the resulting mixture was stirred at room temperature for 2 hours. After washing twice with 20 mL of diethyl ether, the pH of the aqueous layer was adjusted to pH 2 with 3 M HCl. After extraction twice with 20 mL of ethyl acetate, the resulting product was dried using MgSO₄ and then concentrated under reduced pressure to obtain 608 mg of an intermediate 1-((4-fluorophenyl) sulfonyl) piperidine-2-carboxylic acid. The intermediate was used for the next reaction without a further purification process.

After 550 mg of 1-((4-fluorophenyl) sulfonyl) piperidine-2-carboxylic acid was dissolved in 6 mL of THF together with 0.23 mL of N-methylmorpholine-N-oxide (NMO), the resulting solution was stirred at 0° C. After 0.2 mL of ethyl chloroformate was slowly added dropwise, the resulting mixture was stirred at the same temperature for 2 hours. A filtrate obtained after filtering out a solid from the reaction solution was separately stored, and then added dropwise to a solution of 0.2 g of hydroxylamine hydrochloride and 0.4 mL of triethylamine dissolved in 2 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 0.14 g of 1-((4-fluorophenyl) sulfonyl)-N-hydroxypiperidine-2-carboxamide.

¹H-NMR (500 MHz, DMSO-d₆): δ10.63 (s, 1H), 8.76 (s, 1H), 7.82 (m, 2H), 7.42 (m, 2H), 4.32 (m, 1H), 3.62 (m, 1H), 3.46 (m, 1H), 1.80 (m, 1H), 1.77 (m, 1H), 1.51-1.39 (m, 3H), 1.19 (m, 1H)

### (18) Synthesis of T-23 1-((4-((3r,5r,7r)-adamantan-1-yl)phenyl) sulfonyl)-N-hydroxypiperidine-2-carboxamide

After 50 mg of pipecolinic acid was well stirred together with 0.8 mL of THE, 0.9 mL of 1 M Na₂CO₃ was added. 144 mg of 4-(1-adamantyl)benzenesulfonyl chloride was slowly added dropwise at 0° C., and the resulting mixture was stirred at room temperature for 2 hours. After washing twice with 2 mL of diethyl ether, the pH of the aqueous layer was adjusted to pH 2 with 3 M HCl. After extraction twice with 2 mL of ethyl acetate, the resulting product was dried using MgSO₄ and then concentrated under reduced pressure to obtain 90 mg of an intermediate 1-((4-((3r,5r,7r)-adamantan-1-yl)phenyl) sulfonyl) piperidine-2-carboxylic acid. The intermediate was used for the next reaction without a further purification process.

After 50 mg of 1-((4-((3r,5r,7r)-adamantan-1-yl)phenyl) sulfonyl) piperidine-2-carboxylic acid was dissolved in 1 mL of THF together with 15 μL of N-methylmorpholine-N-oxide (NMO), the resulting solution was stirred at 0° C. After 13 μL of ethyl chloroformate was slowly added dropwise, the resulting mixture was stirred at the same temperature for 2 hours. A filtrate obtained after filtering out a solid from the reaction solution was separately stored, and then added dropwise to a solution of 12.9 mg of hydroxylamine hydrochloride and 26 μL of triethylamine dissolved in 1 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 30 mg of 1-((4-((3r,5r,7r)-adamantan-1-yl)phenyl) sulfonyl)-N-hydroxypiperidine-2-carboxamide.

¹H-NMR (500 MHz, DMSO-d₆): δ10.61 (s, 1H), 8.81 (s, 1H), 7.70 (m, 2H), 7.55 (m, 2H), 4.33 (m, 1H), 3.59 (m, 1H), 3.45 (m, 1H), 2.07 (s, 3H), 1.89 (m, 7H), 1.77 (m, 7H), 1.52 (m, 1H), 1.43 (m, 2H), 1.19 (m, 1H)

### (19) Synthesis of T-24 N-hydroxy-1-((4-propylphenyl) sulfonyl) piperidine-2-carboxamide

After 500 mg of pipecolinic acid was well stirred together with 8 mL of THF, 8.9 mL of 1 M Na₂CO₃ was added. 831 μL of 4-n-propylbenzenesulfonyl chloride was slowly added dropwise at 0° C., and the resulting mixture was stirred at room temperature for 2 hours. After washing twice with 10 mL of diethyl ether, the pH of the aqueous layer was adjusted to pH 2 with 3 M HCl. After extraction twice with 20 mL of ethyl acetate, the resulting product was dried using MgSO₄ and then concentrated under reduced pressure to obtain 888 mg of an intermediate 1-((4-propylphenyl) sulfonyl) piperidine-2-carboxylic acid. The intermediate was used for the next reaction without a further purification process.

After 700 mg of 1-((4-propylphenyl) sulfonyl) piperidine-2-carboxylic acid was dissolved in 6 mL of THF together with 272 μL of N-methylmorpholine-N-oxide (NMO), the resulting solution was stirred at 0° C. After 236 μL of ethyl chloroformate was slowly added dropwise, the resulting mixture was stirred at the same temperature for 2 hours. A filtrate obtained after filtering out a solid from the reaction solution was separately stored, and then added dropwise to a solution of 234 mg of hydroxylamine hydrochloride and 470 μL of triethylamine dissolved in 4 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 0.16 g of N-hydroxy-1-((4-propylphenyl) sulfonyl) piperidine-2-carboxamide.

¹H-NMR (500 MHz, DMSO-d₆): δ10.61 (s, 1H), 8.79 (s, 1H), 7.65 (m, 2H), 7.38 (m, 2H), 4.33 (m, 1H), 3.61 (m, 1H), 3.45 (m, 1H), 2.43 (t, 2H, J=7.5 Hz), 1.76 (m, 1H), 1.59-1.38 (m, 6H), 1.16 (m, 1H), 0.90 (t, 3H, J=7.5 Hz)

### (20) Synthesis of T-25 1-((4-acetamidophenyl) sulfonyl)-N-hydroxypiperidine-2-carboxamide

After 500 mg of pipecolinic acid was well stirred together with 8 mL of THF, 8.9 mL of 1 M Na₂CO₃ was added. 1.09 g of N-acetylsulfanilyl chloride was slowly added dropwise at 0° C., and the resulting mixture was stirred at room temperature for 2 hours. After washing twice with 10 mL of diethyl ether, the pH of the aqueous layer was adjusted to pH 2 with 3 M HCl. After extraction twice with 20 mL of ethyl acetate, the resulting product was dried using MgSO₄ and then concentrated under reduced pressure to obtain 935 mg of an intermediate 1-((4-acetamidophenyl) sulfonyl) piperidine-2-carboxylic acid. The intermediate was used for the next reaction without a further purification process.

After 800 mg of 1-((4-acetamidophenyl) sulfonyl) piperidine-2-carboxylic acid was dissolved in 6 mL of THF together with 296 μL of N-methylmorpholine-N-oxide (NMO), the resulting solution was stirred at 0° C. After 258 μL of ethyl chloroformate was slowly added dropwise, the resulting mixture was stirred at the same temperature for 2 hours. A filtrate obtained after filtering out a solid from the reaction solution was separately stored, and then added dropwise to a solution of 256 mg of hydroxylamine hydrochloride and 512 μL of triethylamine dissolved in 4 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 0.18 g of 1-((4-acetamidophenyl) sulfonyl)-N-hydroxypiperidine-2-carboxamide.

¹H-NMR (500 MHz, DMSO-d₆): δ10.60 (s, 1H), 10.32 (s, 1H), 8.78 (s, 1H), 7.74 (m, 2H), 7.68 (m, 2H), 4.30 (m, 1H), 3.61 (m, 1H), 3.45 (m, 1H), 2.09 (s, 3H), 1.76 (m, 1H), 1.51 (m, 1H), 1.39 (m, 3H), 1.16 (m, 1H)

### (21) Synthesis of T-28 1-((4-(tert-butyl)phenyl) sulfonyl)-N-hydroxypyrrolidine-2-carboxamide

After 500 mg of proline was well stirred together with 9 mL of THF, 10 mL of 1 M Na₂CO₃ was added. 1.21 g of 4-tert-butylbenzenesulfonyl chloride was slowly added dropwise at 0° C., and the resulting mixture was stirred at room temperature for 2 hours. After washing twice with 10 mL of diethyl ether, the pH of the aqueous layer was adjusted to pH 2 with 3 M HCl. After extraction twice with 20 mL of ethyl acetate, the resulting product was dried using MgSO₄ and then concentrated under reduced pressure to obtain 860 mg of an intermediate ((4-(tert-butyl)phenyl) sulfonyl) proline. The intermediate was used for the next reaction without a further purification process.

After 700 mg of ((4-(tert-butyl)phenyl) sulfonyl) proline was dissolved in 5 mL of THF together with 272 μL of N-methylmorpholine-N-oxide (NMO), the resulting solution was stirred at 0° C. After 236 μL of ethyl chloroformate was slowly added dropwise, the resulting mixture was stirred at the same temperature for 2 hours. A filtrate obtained after filtering out a solid from the reaction solution was separately stored, and then added dropwise to a solution of 234 mg of hydroxylamine hydrochloride and 470 μL of triethylamine dissolved in 4 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 546 mg of 1-((4-(tert-butyl)phenyl) sulfonyl)-N-hydroxypyrrolidine-2-carboxamide.

¹H-NMR (500 MHz, DMSO-d₆): δ10.66 (s, 1H), 8.94 (s, 1H), 7.78 (m, 2H), 7.64 (m, 2H), 3.91 (m, 1H), 3.30 (m, 1H), 3.13 (m, 1H), 1.88 (m, 1H) 1.75 (m, 1H) 1.66 (m, 1H), 1.46 (m, 1H), 1.31 (s, 9H)

### (22) Synthesis of T-29 1-((4-butoxyphenyl) sulfonyl)-N-hydroxypyrrolidine-2-carboxamide

After 500 mg of proline was well stirred together with 9 mL of THE, 10 mL of 1 M Na₂CO₃ was added. 1.30 g of 4-butoxybenzenesulfonyl chloride was slowly added dropwise at 0° C., and the resulting mixture was stirred at room temperature for 2 hours. After washing twice with 10 mL of diethyl ether, the pH of the aqueous layer was adjusted to pH 2 with 3 M HCl. After extraction twice with 20 mL of ethyl acetate, the resulting product was dried using MgSO₄ and then concentrated under reduced pressure to obtain 1.2 g of an intermediate ((4-butoxyphenyl) sulfonyl) proline. The intermediate was used for the next reaction without a further purification process.

After 1 g of ((4-butoxyphenyl) sulfonyl) proline was dissolved in 7 mL of THF together with 369 μL of N-methylmorpholine-N-oxide (NMO), the resulting solution was stirred at 0° C. After 321 μL of ethyl chloroformate was slowly added dropwise, the resulting mixture was stirred at the same temperature for 2 hours. A filtrate obtained after filtering out a solid from the reaction solution was separately stored, and then added dropwise to a solution of 318 mg of hydroxylamine hydrochloride and 639 μL of triethylamine dissolved in 4 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 370 mg of 1-((4-butoxyphenyl) sulfonyl)-N-hydroxypyrrolidine-2-carboxamide.

¹H-NMR (500 MHz, DMSO-d₆): δ10.65 (s, 1H), 8.93 (s, 1H), 7.76 (m, 2H), 7.12 (m, 2H), 4.07 (t, 2H, J=6.5 Hz), 3.88 (m, 1H), 3.39 (m, 1H), 3.11 (m, 1H), 1.83 (m, 1H), 1.74 (m, 3H), 1.63 (m, 1H), 1.45 (m, 3H), 0.94 (t, 3H, J=8.5 Hz)

### (23) Synthesis of T-32 1-((4-((3r,5r, 7r)-adamantan-1-yl)phenyl) sulfonyl)-N-hydroxypyrrolidine-2-carboxamide

After 50 mg of proline was well stirred together with 0.9 mL of THE, 1 mL of 1 M Na₂CO₃ was added. 162 mg of 4-(1-adamantyl)benzenesulfonyl chloride was slowly added dropwise at 0° C., and the resulting mixture was stirred at room temperature for 2 hours. After washing twice with 2 mL of diethyl ether, the pH of the aqueous layer was adjusted to pH 2 with 3 M HCl. After extraction twice with 2 mL of ethyl acetate, the resulting product was dried using MgSO₄ and then concentrated under reduced pressure to obtain 171 mg of an intermediate ((4-((3r,5r,7r)-adamantan-1-yl)phenyl) sulfonyl) proline. The intermediate was used for the next reaction without a further purification process.

After 130 mg of ((4-((3r,5r,7r)-adamantan-1-yl)phenyl) sulfonyl) proline was dissolved in 2 mL of THF together with 40 μL of N-methylmorpholine-N-oxide (NMO), the resulting solution was stirred at 0° C. After 35 μL of ethyl chloroformate was slowly added dropwise, the resulting mixture was stirred at the same temperature for 2 hours. A filtrate obtained after filtering out a solid from the reaction solution was separately stored, and then added dropwise to a solution of 35 mg of hydroxylamine hydrochloride and 70 μL of triethylamine dissolved in 1 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 90 mg of 1-((4-((3r,5r,7r)-adamantan-1-yl)phenyl) sulfonyl)-N-hydroxypyrrolidine-2-carboxamide.

¹H-NMR (500 MHz, DMSO-d₆): δ10.66 (s, 1H), 8.95 (s, 1H), 7.77 (m, 2H), 7.62 (m, 2H), 3.91 (m, 1H), 3.39 (m, 1H), 3.12 (m, 1H), 2.15 (s, 3H), 1.89-1.75 (m, 13H), 1.65 (m, 1H), 1.50 (m, 1H), 1.16 (m, 1H)

### (24) Synthesis of T-33 N-hydroxy-1-((4-propylphenyl) sulfonyl) pyrrolidine-2-carboxamide

After 500 mg of proline was well stirred together with 9 mL of THE, 10 mL of 1 M Na₂CO₃ was added. 932 μL of 4-n-propylbenzenesulfonyl chloride was slowly added dropwise at 0° C., and the resulting mixture was stirred at room temperature for 2 hours. After washing twice with 10 mL of diethyl ether, the pH of the aqueous layer was adjusted to pH 2 with 3 M HCl. After extraction twice with 20 mL of ethyl acetate, the resulting product was dried using MgSO₄ and then concentrated under reduced pressure to obtain 1 g of an intermediate ((4-propylphenyl) sulfonyl) proline. The intermediate was used for the next reaction without a further purification process.

After 900 mg of ((4-propylphenyl) sulfonyl) proline was dissolved in 6 mL of THF together with 366 μL of N-methylmorpholine-N-oxide (NMO), the resulting solution was stirred at 0° C. After 318 μL of ethyl chloroformate was slowly added dropwise, the resulting mixture was stirred at the same temperature for 2 hours. A filtrate obtained after filtering out a solid from the reaction solution was separately stored, and then added dropwise to a solution of 315 mg of hydroxylamine hydrochloride and 633 μL of triethylamine dissolved in 4 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 665 mg of N-hydroxy-1-((4-propylphenyl) sulfonyl) pyrrolidine-2-carboxamide.

¹H-NMR (500 MHz, DMSO-d₆): δ10.66 (s, 1H), 8.94 (s, 1H), 7.76 (m, 2H), 7.46 (m, 2H), 3.90 (m, 1H), 3.38 (m, 1H), 3.14 (m, 1H), 2.66 (t, 2H, J=9 Hz), 1.83 (m, 1H), 1.75 (m, 1H), 1.65 (m, 2H), 1.44 (m, 2H), 0.89 (t, 3H, J=7.5 Hz)

### (25) Synthesis of T-34 1-((4-acetylphenyl) sulfonyl)-N-hydroxypyrrolidine-2-carboxamide

After 500 mg of proline was well stirred together with 9 mL of THF, 10 mL of 1 M Na₂CO₃ was added. 1.14 g of 4-acetylbenzenesulfonyl chloride was slowly added dropwise at 0° C., and the resulting mixture was stirred at room temperature for 2 hours. After washing twice with 10 mL of diethyl ether, the pH of the aqueous layer was adjusted to pH 2 with 3 M HCl. After extraction twice with 20 mL of ethyl acetate, the resulting product was dried using MgSO₄ and then concentrated under reduced pressure to obtain 958 mg of an intermediate ((4-acetylphenyl) sulfonyl) proline. The intermediate was used for the next reaction without a further purification process.

After 800 mg of ((4-acetylphenyl) sulfonyl) proline was dissolved in 6 mL of THF together with 325 μL of N-methylmorpholine-N-oxide (NMO), the resulting solution was stirred at 0° C. After 283 μL of ethyl chloroformate was slowly added dropwise, the resulting mixture was stirred at the same temperature for 2 hours. A filtrate obtained after filtering out a solid from the reaction solution was separately stored, and then added dropwise to a solution of 280 mg of hydroxylamine hydrochloride and 563 μL of triethylamine dissolved in 4 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 110 mg of 1-((4-acetylphenyl) sulfonyl)-N-hydroxypyrrolidine-2-carboxamide.

¹H-NMR (500 MHz, DMSO-d₆): δ10.72 (s, 1H), 8.97 (s, 1H), 8.15 (m, 2H), 7.97 (m, 2H), 3.95 (m, 1H), 3.43 (m, 1H), 3.13 (m, 1H), 2.65 (s, 3H), 1.86 (m, 1H), 1.72 (m, 2H), 1.48 (m, 1H)

### (26) Synthesis of T-35 N-hydroxy-1-((4-(1-(hydroxyimino)ethyl)phenyl) sulfonyl) piperidine-2-carboxamide

After 1 g of pipecolinic acid was well stirred together with 16 mL of THF, 17.8 mL of 1 M Na₂CO₃ was added. 2.04 g of 4-acetylbenzenesulfonyl chloride was slowly added dropwise at 0° C., and the resulting mixture was stirred at room temperature for 2 hours. After washing twice with 20 mL of diethyl ether, the pH of the aqueous layer was adjusted to pH 2 with 3 M HCl. After extraction twice with 40 mL of ethyl acetate, the resulting product was dried using MgSO₄ and then concentrated under reduced pressure to obtain 1.21 g of an intermediate 1-((4-(1-(hydroxyamino)ethyl)phenyl) sulfonyl) piperidine-2-carboxylic acid. The intermediate was used for the next reaction without a further purification process.

After 1 g of 1-((4-(1-(hydroxyamino)ethyl)phenyl) sulfonyl) piperidine-2-carboxylic acid was dissolved in 10 mL of THF together with 388 μL of N-methylmorpholine-N-oxide (NMO), the resulting solution was stirred at 0° C. After 338 μL of ethyl chloroformate was slowly added dropwise, the resulting mixture was stirred at the same temperature for 2 hours. A filtrate obtained after filtering out a solid from the reaction solution was separately stored, and then added dropwise to a solution of 446 mg of hydroxylamine hydrochloride and 895 μL of triethylamine dissolved in 6 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 0.18 g of N-hydroxy-1-((4-(1-(hydroxyimino)ethyl)phenyl) sulfonyl) piperidine-2-carboxamide.

¹H-NMR (500 MHz, DMSO-d₆): δ11.55 (s, 1H), 10.63 (s, 1H), 8.79 (s, 1H), 7.82 (m, 2H), 7.75 (m, 2H), 4.35 (m, 1H), 3.63 (m, 1H), 3.47 (m, 1H), 3.32 (s, 3H), 1.74 (m, 1H), 1.54 (m, 1H), 1.45 (m, 3H), 1.20 (m, 1H)

### (27) Synthesis of T-36 N-hydroxy-4-methyl-2-(phenylsulfonamido) pentanamide

After 500 mg of leucine was well stirred together with 8 mL of THF, 8.8 mL of 1 M Na₂CO₃ was added. 584 μL of benzenesulfonyl chloride was slowly added dropwise at 0° C., and the resulting mixture was stirred at room temperature for 1 hour. After washing twice with 10 mL of diethyl ether, the pH of the aqueous layer was adjusted to pH 3 with 3 M HCl. After extraction with 20 mL of ethyl acetate, the resulting product was dried using MgSO₄ and then concentrated under reduced pressure to obtain 444 mg of an intermediate (phenylsulfonyl) leucine. The intermediate was used for the next reaction without a further purification process.

After 400 mg of (phenylsulfonyl) leucine was dissolved in 5 mL of THF together with 178 μL of N-methylmorpholine-N-oxide (NMO), the resulting solution was stirred at 0° C. After 155 μL of ethyl chloroformate was slowly added dropwise, the resulting mixture was stirred at the same temperature for 30 minutes. A filtrate obtained after filtering out a solid from the reaction solution was separately stored, and then added dropwise to a solution of 154 mg of hydroxylamine hydrochloride and 308 μL of triethylamine dissolved in 2 mL of DMF. After the resulting mixture was stirred at room temperature for 30 minutes, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (hexane:ethyl acetate=1:1) to obtain 0.11 g of N-hydroxy-4-methyl-2-(phenylsulfonamido) pentanamide.

¹H-NMR (500 MHz, DMSO-d₆): δ10.65 (s, 1H), 8.81 (s, 1H), 8.00 (m, 1H), 7.77 (m, 2H), 7.57 (m, 1H), 7.55 (m, 2H), 3.48 (m, 1H), 1.43 (m, 1H), 1.35-1.20 (m, 2H), 0.72 (d, 3H, J=6.5 Hz), 0.60 (d, 3H, J=7.0 Hz)

### (28) Synthesis of T-40 1-((4-acetamidophenyl) sulfonyl)-N-hydroxypyrrolidine-2-carboxamide

After 1 mg of proline was well stirred together with 18 mL of THE, 20 mL of 1 M Na₂CO₃ was added. 2.44 g of N-acetylsulfanilyl chloride was slowly added dropwise at 0° C., and the resulting mixture was stirred at room temperature for 2 hours. After washing twice with 20 mL of diethyl ether, the pH of the aqueous layer was adjusted to pH 2 with 3 M HCl. After extraction twice with 40 mL of ethyl acetate, the resulting product was dried using MgSO₄ and then concentrated under reduced pressure to obtain 483 mg of an intermediate ((4-acetamidophenyl) sulfonyl) proline. The intermediate was used for the next reaction without a further purification process.

After 450 mg of ((4-acetamidophenyl) sulfonyl) proline was dissolved in 6 mL of THF together with 174 μL of N-methylmorpholine-N-oxide (NMO), the resulting solution was stirred at 0° C. After 152 μL of ethyl chloroformate was slowly added dropwise, the resulting mixture was stirred at the same temperature for 2 hours. A filtrate obtained after filtering out a solid from the reaction solution was separately stored, and then added dropwise to a solution of 200 mg of hydroxylamine hydrochloride and 401 μL of triethylamine dissolved in 1.5 mL of DMF. After the resulting mixture was stirred at room temperature for 2 hours, the DMF was removed by concentration under reduced pressure. After layers were separated by adding ethyl acetate and purified water, the aqueous layer was further extracted twice with ethyl acetate. The organic layer was collected and then concentrated under reduced pressure, and then the resulting solid was purified by column chromatography (5% MeOH in DCM) to obtain 120 mg of 1-((4-acetamidophenyl) sulfonyl)-N-hydroxypyrrolidine-2-carboxamide.

¹H-NMR (500 MHz, DMSO-d₆): δ10.64 (s, 1H), 10.38 (s, 1H), 8.91 (s, 1H), 7.79 (m, 4H), 3.90 (m, 1H), 3.41 (m, 1H), 3.11 (m, 1H), 2.09 (s, 3H), 1.82 (m, 1H), 1.70 (m, 2H), 1.42 (m, 1H)

### Experimental Example

Human monocytic cell line THP-1 cells were seeded into 96-well clear bottom black wells at 1×10\hat{ }⁶ cells/well and treated with 20 ng/ml phorbol 12-myristate 13-acetate (PMA) to induce differentiation into macrophages. After culturing for 3 days, 28 types of amide-based compounds as experimental substances were dissolved in DMSO at 1000× and added to the culture medium so as to be 1 μM or 10 μM, and the cells were treated with the compounds and cultured for 24 hours. In this case, cytochalasin D, which is known to inhibit phagocytosis, was used as a negative control.

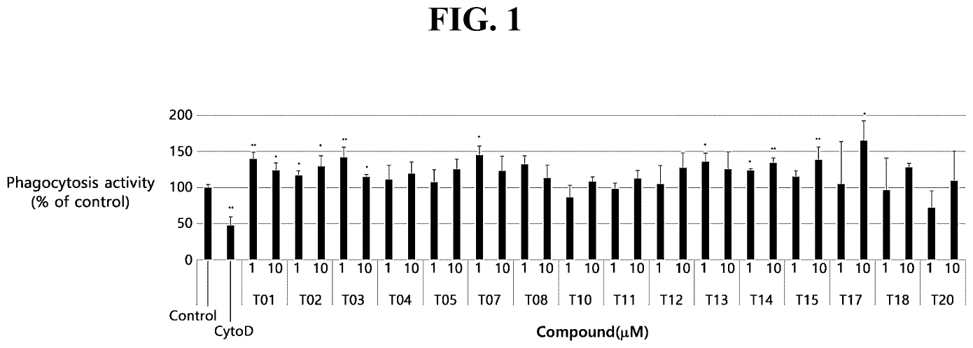

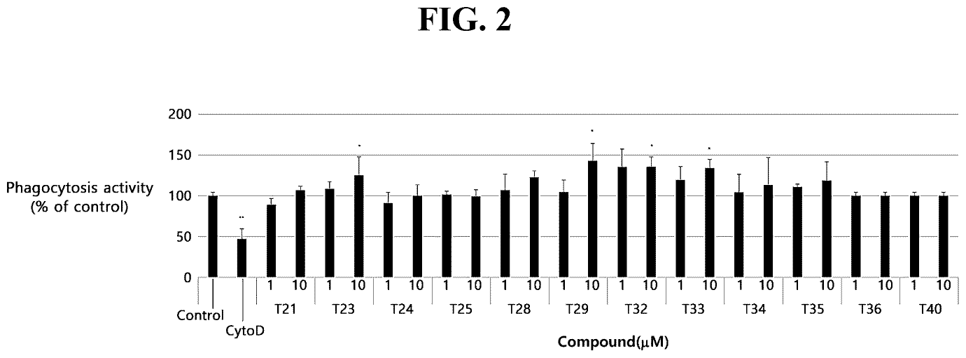

Each well was treated with 80 μl of 0.5 mg/ml pHrodo™ Red S. aureus BioParticles™ Conjugate (ThermoFisher Scientific) and the cells were cultured at 37° C. for 3 hours. Thereafter, fluorescence was measured using a fluorometer (emission 560 nm/excitation 585 nm). Since pHrodo exhibits fluorescence only under acidic conditions, an increase in fluorescent signal indicates an increase in phagocytosis of S. aureus. As a result of measurement by a fluorometer, an increase in phagocytosis of macrophages against S. aureus was confirmed in groups treated with 13 novel amide-based compounds (TO1, T02, T03, T07, T13, T14, T15, T17, T23, T29, and T33) (FIGS. 1 and 2).

We claim:

1. A method for enhancing immunity, the method comprising administering an effective amount of a composition containing, as an active ingredient, a compound represented by the following Chemical Formula 1, a stereoisomer thereof, a pharmaceutically acceptable salt thereof, a hydrate thereof, or a solvate thereof to a subject in need thereof: 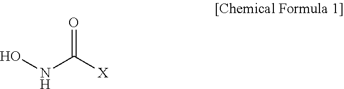
wherein, in Chemical Formula 1,
X is represented by any one of the following Chemical Formulae 1-1 to 1-5, 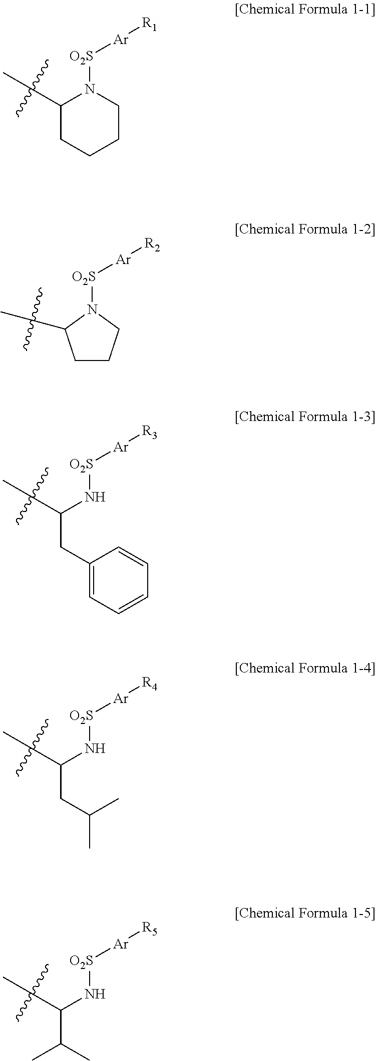
in Chemical Formula 1-1,
Ar is phenyl unsubstituted or substituted with one to three R₁,
R₁ is hydrogen, a phenyl group, a C1-C4 alkoxy group, a C1-C4 alkyl group, a fluoro group, an adamantane group, an acetylamino group, or a hydroxyiminoethyl group,
when there are more than one R₁ residue, all R₁ residues are the same as each other,
in Chemical Formula 1-2,
Ar is phenyl substituted with one R₂,
R₂ is a C1-C4 alkyl group, a C1-C4 alkoxy group, an adamantane group, an acetyl group, or an acetylamino group,
in Chemical Formula 1-3,
Ar is phenyl substituted with one to three R₃ or unsubstituted naphthalene,
R₃ is hydrogen, or a C1-C4 alkyl group or a phenyl group,
when there are more than one R₃ residue, all R₃ residues are the same as each other,
in Chemical Formula 1-4,
Ar is phenyl unsubstituted or substituted with one to three R₄,
R₄ is hydrogen, a phenyl group, a C1-C4 alkyl group, or a C1-C4 alkoxy group,
when there are more than one R₄ residue, all R₄ residues are the same as each other,
in Chemical Formula 1-5,
Ar is phenyl substituted with one to three R₅,
R₅ is a C1-C4 alkyl group or a phenyl group, and
when there are more than one R₅ residue, all R₅ residues are the same as each other.

2. The method of claim 1, wherein the compound represented by Chemical Formula 1 is any one selected from the following compounds:
N-hydroxy-1-(phenylsulfonyl) piperidine-2-carboxamide,
1-([1, l′-biphenyl]-4-ylsulfonyl)-N-hydroxypiperidine-2-carboxamide,
1-((4-(tert-butyl)phenyl) sulfonyl)-N-hydroxypiperidine-2-carboxamide,
1-((4-butoxyphenyl) sulfonyl)-N-hydroxypiperidine-2-carboxamide,
N-hydroxy-1-(mesitylsulfonyl) piperidine-2-carboxamide,
2-([1,1′-biphenyl]-4-sulfonamido)-N-hydroxy-3-phenylpropanamide,
2-((4-(tert-butyl)phenyl) sulfonamido)-N-hydroxy-3-phenylpropanamide,
N-hydroxy-3-phenyl-2-((2,4,6-trimethylphenyl) sulfonamido) propanamide,
N-hydroxy-2-(naphthalene-2-sulfonamido)-3-phenylpropanamide,
2-([1,1′-biphenyl]-4-sulfonamido)-N-hydroxy-4-methylpentanamide,
2-((4-(tert-butyl)phenyl) sulfonamido)-N-hydroxy-4-methylpentanamide,
2-((4-butoxyphenyl) sulfonamido)-N-hydroxy-4-methylpentanamide,
N-hydroxy-4-methyl-2-((2,4,6-trimethylphenyl) sulfonamido) pentanamide,
2-([1,1′-biphenyl]-4-sulfonamido)-N-hydroxy-3-methylbutanamide,
2-((4-(tert-butyl)phenyl) sulfonamido)-N-hydroxy-3-methylbutanamide,
N-hydroxy-3-methyl-2-((2,4,6-trimethylphenyl) sulfonamido) butanamide,
1-((4-fluorophenyl) sulfonyl)-N-hydroxypiperidine-2-carboxamide,
1-((4-((3r,5r,7r)-adamantan-1-yl)phenyl) sulfonyl)-N-hydroxypiperidine-2-carboxamide,
N-hydroxy-1-((4-propylphenyl) sulfonyl) piperidine-2-carboxamide,
1-((4-acetamidophenyl) sulfonyl)-N-hydroxypiperidine-2-carboxamide,
1-((4-(tert-butyl)phenyl) sulfonyl)-N-hydroxypyrrolidine-2-carboxamide,
1-((4-butoxyphenyl) sulfonyl)-N-hydroxypyrrolidine-2-carboxamide,
1-((4-((3r,5r, 7r)-adamantan-1-yl)phenyl) sulfonyl)-N-hydroxypyrrolidine-2-carboxamide,
N-hydroxy-1-((4-propylphenyl) sulfonyl) pyrrolidine-2-carboxamide,
1-((4-acetylphenyl) sulfonyl)-N-hydroxypyrrolidine-2-carboxamide,
N-hydroxy-1-((4-(1-(hydroxyimino)ethyl)phenyl) sulfonyl) piperidine-2-carboxamide,
N-hydroxy-4-methyl-2-(phenylsulfonamido) pentanamide, and
1-((4-acetamidophenyl) sulfonyl)-N-hydroxypyrrolidine-2-carboxamide.

3. The method of claim 1, wherein the compound represented by Chemical Formula 1 is any one selected from the following compounds:
N-hydroxy-1-(phenylsulfonyl) piperidine-2-carboxamide,
1-([1,1′-biphenyl]-4-ylsulfonyl)-N-hydroxypiperidine-2-carboxamide,
1-((4-(tert-butyl)phenyl) sulfonyl)-N-hydroxypiperidine-2-carboxamide,
2-([1,1′-biphenyl]-4-sulfonamido)-N-hydroxy-3-phenylpropanamide,
2-((4-(tert-butyl)phenyl) sulfonamido)-N-hydroxy-4-methylpentanamide,
2-((4-butoxyphenyl) sulfonamido)-N-hydroxy-4-methylpentanamide,
N-hydroxy-4-methyl-2-((2,4,6-trimethylphenyl) sulfonamido) pentanamide,
2-([1,1′-biphenyl]-4-sulfonamido)-N-hydroxy-3-methylbutanamide,
1-((4-((3r,5r,7r)-adamantan-1-yl)phenyl) sulfonyl)-N-hydroxypiperidine-2-carboxamide,
1-((4-butoxyphenyl) sulfonyl)-N-hydroxypyrrolidine-2-carboxamide, and
N-hydroxy-1-((4-propylphenyl) sulfonyl) pyrrolidine-2-carboxamide.

4. The method of claim 1, wherein the compound represented by Chemical Formula 1 is derived from an amino acid.

5. The method of claim 1, wherein the enhancing immunity is activating a phagocytosis of macrophages.

6. The method of claim 1, wherein the enhancing immunity is activating a phagocytosis of macrophages against S. aureus.

7. The method of claim 1, wherein the enhancing immunity is strengthening the skin barrier.

8. The method of claim 1, wherein the enhancing immunity is suppressing skin inflammation.

9. The method of claim 1, wherein the composition is a cosmetic composition.

10. The method of claim 1, wherein the composition is a skin external preparation.

11. The method of claim 1, wherein the composition is a food composition.

12. The method of claim 1, wherein the composition is a pharmaceutical composition.

13. The method of claim 12, wherein the enhancing immunity is preventing, ameliorating, or treating skin inflammation.

14. The method of claim 1, wherein a content of the active ingredient is 0.01 μM to 100 mM based on a total volume of the composition.

15. The method of claim 1, wherein a daily application amount of the active ingredient is 1 to 1,000 mg/kg.

16. The method of claim 1, wherein cells are treated with the active ingredient at a concentration of 1 to 100 μM.
